(19) United States
(12) Patent Application Publication
(10) Pub. No.: US 2015/0147367 A1
(43) Pub. Date: May 28, 2015

(54) METHOD FOR NANOCAPSULATION OF HYDROPHOBIC COMPOUNDS AND COMPOSITIONS THEREOF

(71) Applicant: Tarbiat Modaress University, Tehran (IR)

(72) Inventors: Soleiman Abbasi, Tehran (IR); Soheila Ghasemi, Tehran (IR)

(73) Assignee: Tarbiat Modaress University, Tehran (IR)

(21) Appl. No.: 14/493,938

(22) Filed: Sep. 23, 2014

Related U.S. Application Data
(60) Provisional application No. 61/880,988, filed on Sep. 23, 2013.

(51) Int. Cl. A61K 9/51; A61Q 19/00; A61K 8/11; A61K 8/64; A23L 1/00; A23L 1/30

(52) U.S. Cl. CPC A61K 9/5192; A23L 1/0032; A23L 1/3008; A61K 9/5169; A61K 8/11; A61K 8/64; A61Q 19/00; A23V 2002/00; A61K 2800/413; A61K 2800/10
USPC 424/401; 426/238; 426/96; 424/400

(57) ABSTRACT

Nanoencapsulation of hydrophobic compounds using native casein micelles by means of PH changes and ultrasonication.

### CROSS REFERENCE TO RELATED APPLICATION

The present invention claims priority from pending U.S. Provisional Patent Application Serial No. 61/880,988, filed Sep. 23, 2013, entitled “Nanocapsulation of hydrophobic compounds using native casein micelles by means of pH changes and ultrasonication,” the subject matter of which is incorporated by reference herein in its entirety.

### SPONSORSHIP STATEMENT

This application has been sponsored by the Iranian Nanotechnology Initiative Council, which does not have any rights in this application.

### TECHNICAL FIELD

The present invention relates to the delivery of hydrophobic active compounds via beverages and food. Additionally, the present invention provides nanocapsulation of hydrophobic active compounds in natural casein micelles, stabilization and protection of nanocapsulated hydrophobic active compounds, and methods of producing same.

### BACKGROUND OF THE INVENTION

With the growing public realization of the importance of the role food plays in disease prevention, new technologies (e.g., micro or nanoencapsulation) have been introduced to enrich foods with health-promoting ingredients and producing so-called functional foods. In this regard, the use of oils containing essential fatty acids, such as rapeseed, soy bean, and fish oils, are one of the relatively major challenges in low-fat and fat-free foods since the ω-3 fatty acids have healthy characteristics, such as reducing the risks of cardiovascular diseases and cancers, as well as having a key role in brain development.

However, due to their physical and oxidative instability, these compounds need stabilizing in an aqueous medium, as well as protection against destructive factors, including oxygen and pro-oxidants. Thus, encapsulation is a way to protect these sensitive compounds against such external factors without requiring the addition of antioxidants. Proteins, such as caseins, are a common type of biopolymers that, because to their favorable textural, flavor and functional characteristics are used in the stabilisation of emulsions, as well as encapsulation of hydrophobic compounds.

Casein micelles are globular colloids (d=50-500 nm, 150 nm on average) with a high molecular mass (10⁶-10⁹ Dalton) made of of αs₁, αs₂, β, and κ caseins (1:4:1:4), respectively. Regarding the internal structure of micelles, caseins are mostly held together by hydrophobic interactions and bridging of calcium-phosphate with serine-phosphate residues.

Studies have shown that caseins lack any rigid secondary (both α-helix and β-pleated sheets) structure, and have a significant number of hydrophobic residues, for which they adsorb strongly at the droplet surface. Furthermore, caseins can form a thicker interfacial layer (about 10 nm) around fat droplets, as compared with whey (1-2 nm) proteins. These characteristics are chief reasons for their high encapsulation efficiency. As for the digestibility, caseins have an open tertiary structure, due to high proline content, which results in easier access of gastric proteases, as well as release of the encapsulated substances in the stomach, such that in some studies this feature has been utilized for oral drug delivery for gastric diseases.

Casein, as a wall material, is usually used for encapsulation (emulsification and homogenization followed by spray drying) of hydrophobic substances. But, during this process, it normally loses its original micellar structure, as well as the aforesaid functional characteristics. Moreover, it has been found that it is likely that producing bigger microcapsules would impair product smoothness and that caseins do not have high oxidative stability. is

A Millard reaction (casein and carbohydrate reaction) is another method for encapsulation, as noted in the prior art. For example, U.S. Patent Application Publication No. US20070218125 describes a micro encapsulation material for use with storage unstable, therapeutic and nutritional agents that release the therapeutic and nutritional agents in predetermined locations in the gastro intestinal tract, in which the microencapsulation material is formed by combining a food grade treated carbohydrate with a water soluble food grade protein. The use of the aforementioned Millard reaction for encapsulation, however, wastes amino acids (e.g., lysine), and increase the resistance of the Millard product to digestive enzymes, as well as to reduce the nutritious characteristics of proteins. Furthermore, it is not still clear yet whether the improvement on oxidative stability in this process is due to the changes in the morphology of the produced capsules or due to the anti-oxidative characteristics of the compounds produced by Millard reaction.

Due to the abovementioned problems and issues, recent research has attempted to entrap the hydrophobic substances through re-assembling the casein micelle using calcium, phosphate, and citrate. The re-assembled casein micelles (rCM), due to their having aromatic side groups and double bonds, have been found to have a good protective effect on vitamin D2 against ultraviolet radiation. In fact, it is likely that casein, because of absorbing or scattering much of the light, prevents light from reaching the hydrophobic core.

A rather inefficient system is set forth in Patent Application No. WO2007122613A1, which describes a system based on re-assembled casein micelles (rCM) for the delivery of hydrophobic biologically active compounds in food and beverages and the method for the preparation of a re-assembled casein micelle, in which a cosolvent solution is added in a casein solution and then mixed with a source of citrate ions, a source of phosphate ions and a source of calcium ions. All these successive steps for making casein micelles have resulted in efficiency of merely 27%.

In some other studies, a low density protein (CMC=0.05-0.2 V/W) was used to increase the possibility of interaction between the hydrophobic substance and the hydrophobic moiety of the casein. It was found that in such concentration, the monomers of protein are often dominant, and their hydrophobic parts have no connection with other proteins. Therefore, the probability of interaction with the hydrophobic substance increases. In fact, the more surface area available for binding, it has been determined that the more interaction will be made.

Up to now, natural casein micelles have not ever been used for encapsulating hydrophobic compounds with maintaining their structural features. To make this happen, requires increasing the accessible hydrophobic areas, which are normally buried in interior parts of micelles with little chance for bonding with hydrophobic compounds. It is known in the art that at higher pH, the structure of casein micelles becomes expanded and wider due to electrostatic repulsion between protein monomers. Furthermore, it has been found that an alkaline condition (pH-8) affects the secondary structure of casein, and that this change can increase its interaction with vitamin D₂. In addition, it has been reported that sonication at high pH values (6.6-12) can significantly affect the particle size distribution via breaking non-peptide bonds of the re-assembled casein.

Based on the abovementioned knowledge in this art, the present invention elucidates the effect of the coincident use of alkaline pH and sonication on exposure of hydrophobic areas of protein in order to be used for encapsulation of the PUFA oils inside the natural casein micelles. For simplification, the following abbreviations are used: P (pH change), U (sonication), UP (sonication followed by pH change), PUP (pH change, sonication followed by pH change), PU (pH change followed by sonication) were used.

There is, therefore, a present need for improves methodologies, techniques and compositions for use in drug applications, as vitamins and nutraceuticals, food technology, cosmetics and many other usages that involve the efficient encapsulation of a hydrophilic composition within a casein micelle or like material.

These and many other objects are met in various embodiments of the present invention, offering significant advantages over the known prior art and consequent benefits in the extraction techniques.

### SUMMARY OF THE INVENTION

The present invention is directed generally to the nanoencapsulation of hydrophobic compounds in casein micelles by means of PH changes and ultrasonication. For embodiments of the present invention involving milk, this results in the creation of natural casein micelle nanocapsules with high encapsulating efficiency, while maintaining natural casein structure and morphological features.

In one embodiment of the present invention, with an increase in pH (from 6.7 up to 11), the turbidity of skim milk decreased. Moreover, with an increase in pH (from 6.5 up to 8), the size of casein micelle particles (as measured by Dynamic Light Scattering technique), as well as their physical stability and encapsulation capability increased. The synchronization of the particle size increase with turbidity decrease, as well as the increase in the encapsulation efficiency, is related to the loose-fitting structure of natural casein micelles, due to the electrostatic repulsion between the casein molecules, which ultimately leads to an increase in availability of interior hydrophobic areas. As a result of these characteristics, the hydrophobic compounds (e.g., oils, unsaturated fatty acids) can be incorporated into the most interior part of natural casein micelles, where with further pH changes (from 8 to 6.7) they can be encapsulated therein as nanoparticles.

According to currently preferred embodiments of the present invention, for best results in encapsulation efficiency an ultrasound treatment step is employed, and the significant role of its exposure time and order in the process steps arrangement in the degree of effectiveness on the encapsulating efficiency of casein micelles wherein PUP(pH changing, ultrasonication, pH changing) process demonstrated a higher encapsulation efficiency compared to PU (pH change, ultrasonication) or UP (ultrasonication, pH change) processes, a marked leap over the known prior art.

The present invention provides a system, method and technique based on the usage of natural casein micelles for the delivery of hydrophobic compounds, as encapsulated compounds, which can be selected from hydrophobic nutrients, nutraceuticals, vitamins, and drugs. The present invention utilizes only natural, safe and nontoxic components, and thereby maintains and preserves the natural structure and functions of the casein micelles.

In some embodiments the casein micelles employed in the present invention range from about 200 nm to about 350 nm in diameter. In other embodiments the average diameter of the casein micelles range from about 250 nm to about 350 nm.

In the present invention, the excellent oxidative stability of the produced casein micelle nanocapsules against UV light (for 18 hours) means that the compositions and products produced pursuant to the teachings of the present invention may be employed in a wide range of usages.

Further embodiments and the full scope of applicability of the present invention will become apparent from the detailed description given hereinafter.

### BRIEF DESCRIPTION OF THE DRAWINGS

While the specification concludes with claims particularly pointing out and distinctly claiming the subject matter that is regarded as forming the present invention, it is believed that the invention will be better understood from the following description taken in conjunction with the accompanying DRAWINGS, where like reference numerals designate like structural and other elements, in which:

FIG. 1 generally shows illustrates the Influence of skimmed milk pH on its absorbance (λ=286 nm, temperature 30° C.) pursuant to the teachings of the present invention;

FIG. 2 shows a illustrates the size distribution of skimmed milk at pH 6.7 (♦), pH 8.0 ( ) and pH 9.0 (Δ) pursuant to the principles of the instant invention;

FIG. 3 shows Illustrates the Effect of oil content on encapsulation efficiency (EE) of natural casein micelles prepared by P method. Different small letters show significant differences (P <0.01)pursuant to the teachings of the present invention;

FIG. 4 shows a the comparison between the effect of ultracentrifuge (60,000 g) and isoelectric point precipitation methods on encapsulation efficiency value pursuant to the teachings of the present invention;

FIG. 5 shows Illustrates the effect of different treatments on encapsulation efficiency (EE) of natural casein micelles (sonicated at constant amplitude 25%, and oil content 0.68 w/v %);

FIG. 6 illustrates the influence of UV radiation (78 mW/cm² and λ=200-290 nm) on peroxide value of rapeseed oil (0.54%) encapsulated (PUP) by natural casein micelle nanocapsules and a control without any treatment; and

FIG. 7 illustrates several microscopic image results by different treatments (P, PU, UP, PUP) on particles size distribution of natural casein micelles nano-capsules containing rapeseed oil (0.54 w/v %), where FIG. 7A is a microscopic image when no treatment is used and the Z average is 242 nm; where FIG. 7B shows a microscopic image when the method of treatment is skim milk+oil and only PH changes (pH was changed from 6.7 to 8.0 and vice versa) and Z average is 346 nm; and where FIG. 7C is a microscopic image when the treatment method is skim milk+oil PUP (pH was changed from 6.7 to 8.0, sonicated for 2 min at amplitude 50%, then pH changed from 8.0 to 6.7) with Z average of 245 nm.

### DETAILED DESCRIPTION OF THE INVENTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

The preparation of Nanocapsules

### Materials

In practicing the principles of the present invention, fresh cow milk (protein 3%, casein 2.2-2.4%, and fat 3.5%), crude rapeseed and soy bean oils were purchased from local suppliers. Crude sardine fish oil (fished from Persian Gulf) was extracted using solvents. Chemicals such as HCl (37%), sodium hydroxide, petroleum ether, diethyl ether and ammonium were purchased.

### Purification of Oils

In order to eliminate the gums and impurities, the crude oils (fish or vegetable oils) were mixed with water (5:1), shaken (1 min) and centrifuged (10000 g, 4 ° C. for 15 min). Eventually, the supernatant (oil phase) was mixed with hexane (1:3) and filtered. In the next stage, the solvent was vaporized and the purified oils were kept in capped containers at 4° C.

Incorporation of Oil into Natural Casein Micelles (Nanoencapsulation)

In an experiment implementing the principles of the present invention, the pH of skim milk (fat content ˜0.08%) was increased to 8 (NaOH, 1 N), while stirred (1200 rpm). Then, the milk was kept for 15-20 min in order to complete any interaction between hydroxyl ions and caseins. In the next step, oil (0.37, 0.54, 0.68, 0.78, and 1.2 w/v %) was slowly added, and after about 20 min the pH was adjusted (pH=8), and after another 20 min the pH was reduced (˜6.7 using HCl, 0.1 N). This sample was called P. Similar changes were done on a control sample, with the exception of changing the pH, and the physical stability was investigated.

For sonication purposes, the samples were treated [Freq=25 kHz, 600 Watt, high gain sonotrode (d=19 mm, amplitude=0-100%)] at different exposure times (up to 4 min) in four states, namely UP, PUP, PU, and U (only 4 min exposure time). In the case of UP and PUP, the samples were treated 20 min after adding oil, while in case of PU, it was done after readjusting the pH to its natural level. It should be understood that a comparison of the effects of ultrasound intensity (amplitude 25, 50, and 100%) and exposure time (0.5, 1, 2, and 4 min) on encapsulation efficiency was done on PUP samples.

### Turbidity Measurement

The turbidity of samples treated by ultrasound and pH changes was measured, approximately 20 minutes after pH setting, using a spectrophotometer at 30° C.

### Encapsulation Efficiency (EE)

In order to measure the encapsulation efficiency, the total amount of oil and extractable oil were measured. Then, encapsulation efficiency was calculated through the following equation:

$$EE = \frac{total oil - extractable oil}{total oil} \times 10 \qquad Equation 1$$

The total oil content was measured by modified Rose Gottlieb method (sample size=50 ml). For calculating the extractable oil (non-encapsulated oil), two different procedures were used. In the first one, based on gravimetrical precipitation, nano-capsules (prepared by P and PUP methods) were precipitated (ultracentrifuge in 25° C. at 60,000 g for 2 hours) and supernatant was mixed with few drops of Congo red and ammunia (10 w/v %). Then, the free oil was extracted using petroleum and diethyl ethers, as is understood in the art.

In the second procedure, based on the precipitation of nano-capsules at isoelectric pH, the pH of the P samples (containing 0.54, 0.68, 0.78, and 1.2 w/v rapeseed oil) were reduced to 4.5 by adding HCl (0.1 N) followed by centrifugation (4000 g, 5 min, 25° C.).

### Measurement of Saponification

Due to the alkaline pH, the possibility of soap formation was a concern. Therefore, the potential amount of soap was assessed based on a simple procedure. To this end, some oil (0.54 w/v %) was added to plain and P skim milks then their total oil contents were measured (Rose Gottlieb method) and any difference attributed to the saponification.

### The Size Distribution

The average diameter, the size distribution, and zeta potential of samples were measured using a particle size analyzer. For measuring zeta potential, the samples were diluted (5 times) by de-ionized water just before assessment.

### The Protective Effect of Nanoencapsulation

Pasteurized (72 ° C., 20 sec) samples (including P, UP, PUP, PU, U and control) were placed in petri dishes (20 ml, d=9.5 cm) and exposed to UV light (UVC over the range 200-290 nm, energy=77.89 mW/cm2), for 1, 3, 6, 12, and 18 h. Then, the respective peroxide values were measured using a modified method.

### Effect of pH on Turbidity and Particle Size Distribution

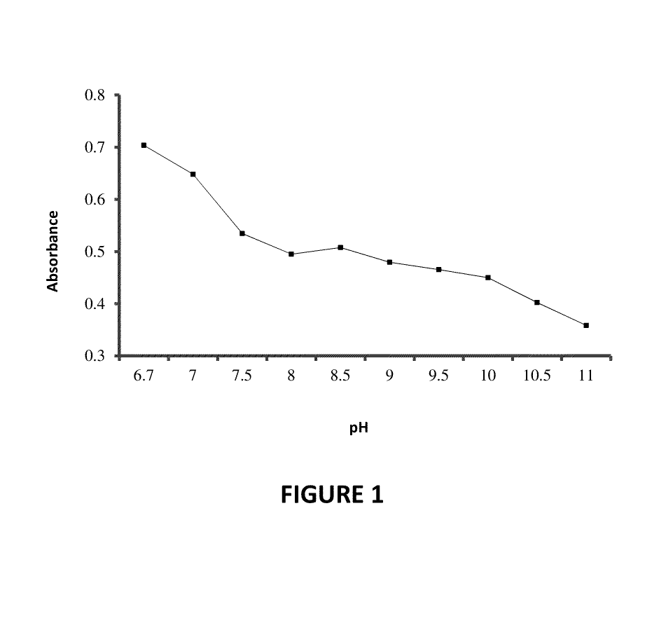

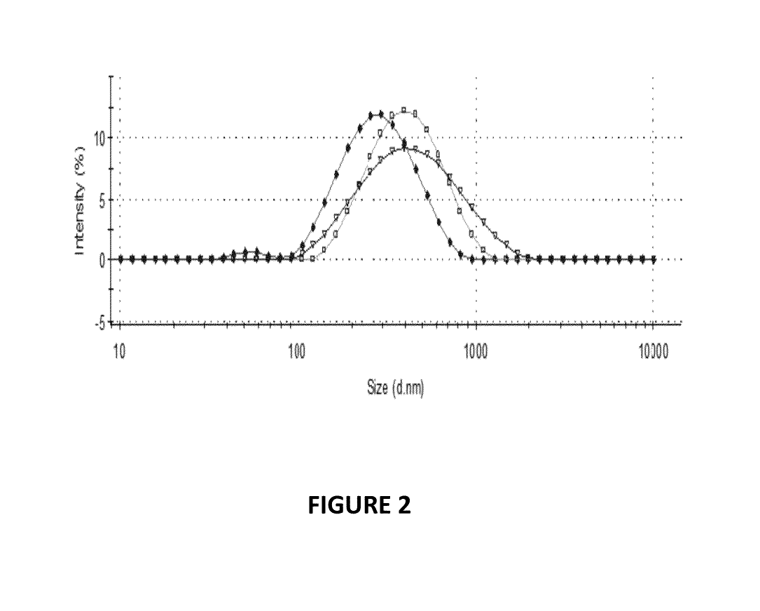

Based on the findings, the turbidity (λ=286 nm) of skim milk dispersion decreased as pH increased (6.7 to 11.0). This change was marked over the range 6.7 to 8.0, while it had a modest slope above this range, as illustrated and described in connection with FIG. 1 of the DRAWINGS. In addition, over this range (6.7 to 8.0) the size of the skim milk particles increased (from 306 to 421 nm). It is noteworthy that further decreases of pH to its original value (even after sonication) led to the reduction of particle sizes. These findings confirm that the changes (turbidity and size) caused by alkaline pH were reversible, as illustrated and described in connection with FIG. 2 of the DRAWINGS. In fact, with pH increase, casein molecules gain more negative charges, as well as stronger electrostatic repulsion, which results in larger and looser structures. However, the attractive forces are still sufficient to maintain the micellar integrity of casein particles even at strongly alkaline pHs.

### The Influence of pH on EE of Natural Casein Micelle Nanocapsules

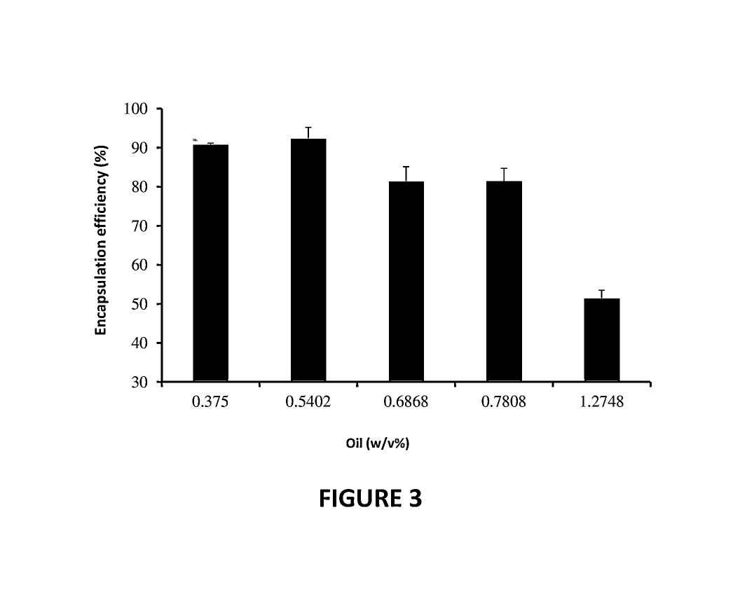

The encapsulation efficiency (EE) of samples treated by P method showed inverse proportionality with oil content, where with increasing the oil proportion, the physical stability and EE was reduced, as illustrated and described in connection with FIG. 3 of the DRAWINGS. Moreover, the oil type (crude soy bean, rapeseed, or fish oil) had no significant effect on the physical stability. Therefore, in the next experiments, the rapeseed oil (as a model hydrophobic compound) was used. It should be understood that at higher pH (>8) the molecular structure becomes looser and the oil incorporation happens via recently-available hydrophobic patches while with its further decrease (down to 6.7); the oil droplets can be entrapped within casein micelle. In fact, with an increase of the accessible hydrophobic area, there would be more possibilities for connection. In addition, the wall material is probably insufficient for covering hydrophobic cores (oil particles) in a high proportion of oils.

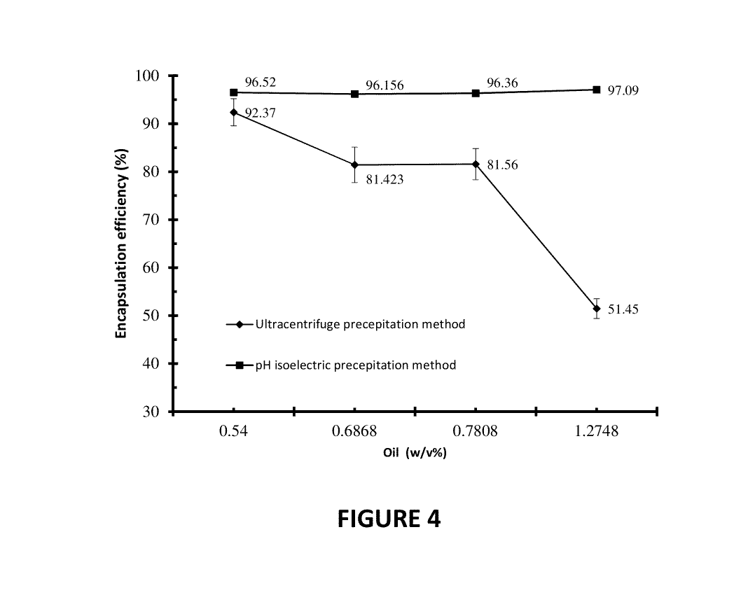

In addition, the comparison of two EE measuring methods (precipitation by ultracentrifuge and precipitation in isoelectric pH) showed that in the case of the former method (ultracentrifuge procedure), the EE reduced as the oil content increased, while regarding latter method (isoelectric pH), no considerable difference was observed between samples containing various proportions of oil, as illustrated and described in connection with FIG. 4 of the DRAWINGS. In other words, there was a direct relation between physical stability and EE in an ultracentrifuge procedure, while in precipitation with the isoelectric pH method such a relationship was not seen. It seems that precipitation of nano-capsules in isoelectric pH leads to the precipitation of casein micelles, as well as those oil droplets which have had a weak connection with protein particles. On the other hand, it is believed that in acidic conditions (about 3.7 and 4) the oil droplets, which are covered by proteins can easily exhibit behavior similar to protein particles. 20

### The Influence of Sonication on EE of Natural Casein Micelle Nanocapsules

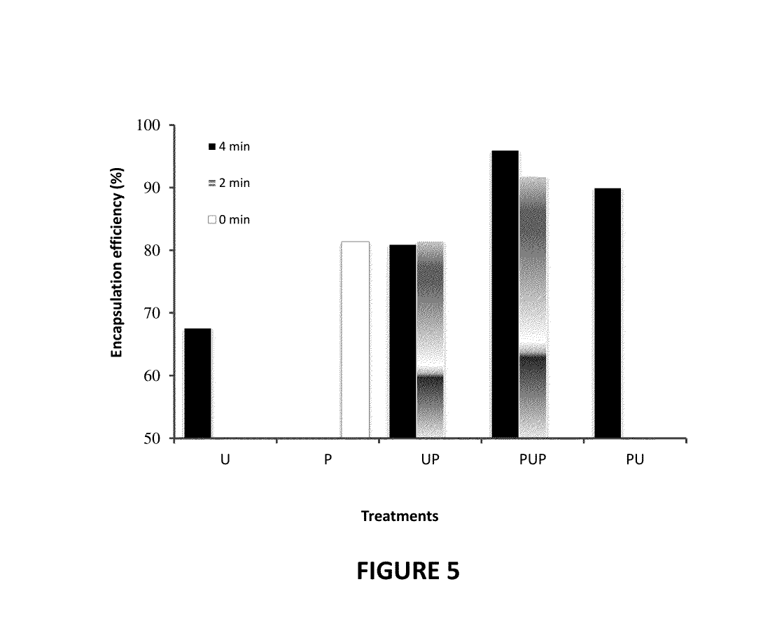

As illustrated and described in connection with FIG. 5 of the DRAWINGS, the application of ultrasound treatment (PUP method in sample containing %0.68 rapeseed oil) significantly increased the encapsulation efficiency in comparison to the aforementioned U, PU, and UP methods. In general, the effectiveness order of the abovementioned methods on encapsulation efficiency can be summarized as follows:

PUP₄>PUP₂>PU₄>UP₄=UP₂=P>U

where the subscripts refer to the exposure (minute) to sonication at constant amplitude (25%)

In the case of PUP, the reduction of oil particles size (due to sonication) has probably been synchronized with the expansion of casein micelle structure (because of the pH increase) and this has raised the possibility of interaction between oil particles and hydrophobic areas of casein micelles. Moreover, the further treatment with ultrasound at higher pH values more likely increases the relative sono-disruption and breakage of the non-peptide bonds in casein micelles due to the looser structure of casein at alkaline conditions. As a result, additional hydrophobic areas of casein micelle might be exposed to oil droplets, where with the further decrease of pH to its initial level (6.7), the oil droplets are trapped within the casein micelles.

TABLE 1 set forth hereinbelow shows the impact of different amplitudes and exposure times of sonication (PUP method) on the EE of skim milk containing 0.68% rapeseed oil. As can be seen, the highest encapsulation efficiency belongs to the sample that was sonicated for 2 min at amplitude of 50%; therefore, this condition was used and considered for the further steps set forth herein.

TABLE 1
Effect of sonication conditions (amplitude and exposure time) on
encapsulation efficiency (EE %) of natural casein micelles prepared
by PUP (skimmed milk containing 0.68% w/v rapeseed oil).

| Exposure Time | Amplitude (%) | | |
|---|---|---|---|
| (min) | 25 | 50 | 100 |
| 0.5 | — | — | 89.30 ^{b} |
| 1.0 | 79.93 ^{c} | 94.90 ^{a} | 91.08 ^{b} |
| 2.0 | 91.78 ^{b} | 96.33 ^{a} | 90.27 ^{b} |
| 4.0 | 96.00 ^{a} | 90.91 ^{b} | — |

Different small letters represent significant differences at 95% (P < 0.05)

### Effect of Alkaline pH on Saponification

The comparison of the total oil content of control skim milk (U method, containing 0.54 w/v % of rapeseed oil) with one which prepared with pH changes (P method, containing similar amount of rapeseed oil) revealed that their total fat contents after treatment were 0.540 and 0.536 w/v %, respectively. As can be seen, the difference was extremely small and negligible. Therefore, it can be concluded that alkalization of milk does not lead to saponification.

### Effect of Treatments on Size Distribution and Zeta Potential

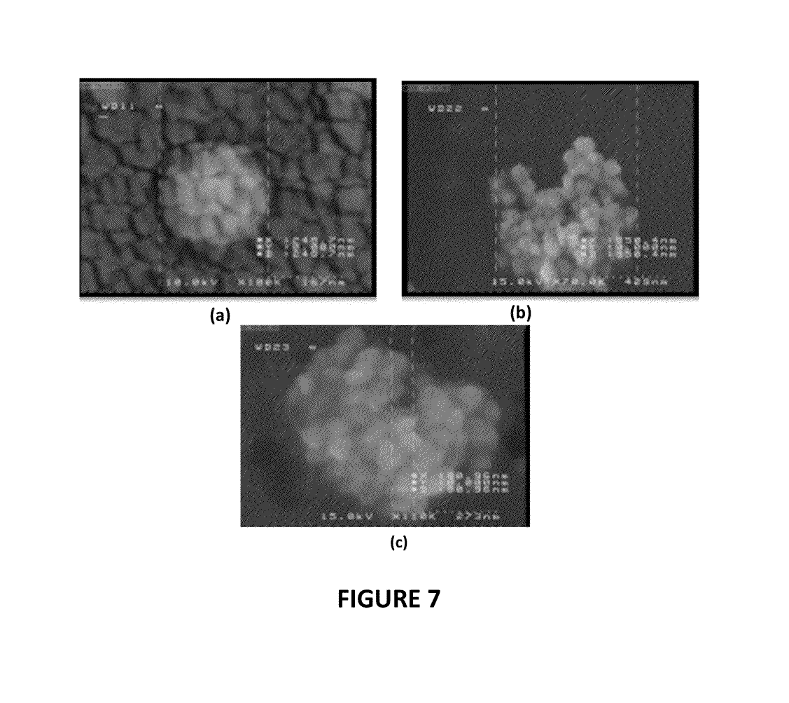

TABLE 2 set forth hereinbelow and as illustrated and described in connection with FIG. 7 of the DRAWINGS, particularly FIGS. 7A, 7B and 7C, these show the size distribution of particles in the plain skim milk, as well as skim milks enriched with oil, which were treated by P and PUP methods. It can be seen that the mean diameter of particles in skim milk is about 306 nm, while in the P sample this value increased up to 437 nm. This is a clear indication of the encapsulation of added oil by casein micelles. In contrast to the aforesaid PU and UP methods, in the PUP method the distribution pattern was monomodal, where its mean diameter was around 312 nm, which was quite similar to plain skim milk. These findings clearly show the very interesting capability of casein micelles in nanoencapsulation of added oil under the combined assistance of pH change and sonication, where neither of these treatments individually were capable of nanoencapsulation.

TABLE 2
Effect of different treatments (P, PU, UP, PUP) on particles
size distribution of natural casein micelles nano-capsules
containing rapeseed oil (0.54 w/v %)

| Treatment | Z-Average (nm) | PDI |
|---|---|---|
| Skim milk | 242 | 0.192 |
| Skim milk + oil | 330 | 0.207 |
| (without treatment) | | |
| Skim milk + oil | 346 | 0.265 |
| P (pH was changed from 6.7 to 8.0 and | | |
| vice versa) | | |
| Skim milk + oil | 238 | 0.203 |
| UP (sonicated for 2 min at Amplitude | | |
| 50%, then pH was changed from 6.7 to | | |
| 8.0 and vice versa) | | |
| Skim milk + oil | 245 | 0.199 |
| PUP (pH was changed from 6.7 to 8.0, | | |
| sonicated for 2 min at Amplitude 50%, | | |
| then pH changed from 8.0 to 6.7) | | |
| Skim milk + oil | 248 | |
| PU (pH was changed from 6.7 to 8.0 and | | |
| vice versa, then sonicated for 2 min at | | |
| Amplitude 50%) | | |

Apart from particle size measurements, the zeta potential measurements on plain skim milk and the one containing rapeseed oil (0.54%) which treated by the PUP method were also clearly confirmed (˜−19 my in both cases) that natural casein micelles substantially maintained their structural features during PUP treatment, as no change was observed in the electrical charge of casein micelles.

### Oxidative Stability of Oils Encapsulated in Natural Casein Micelle Nanocapsules

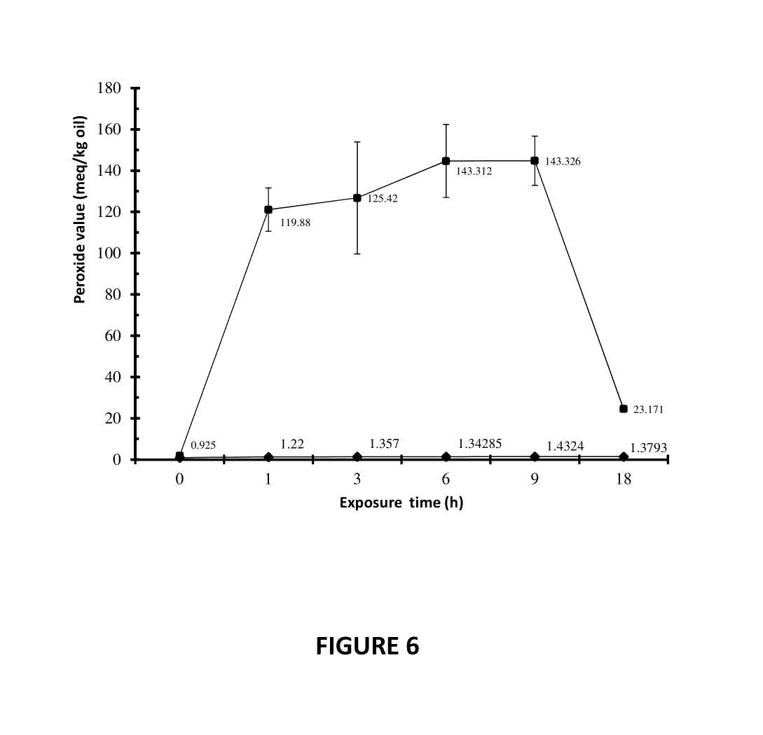

As illustrated and described in connection with FIG. 6 of the DRAWINGS, the effect of exposure to UV for 18 hours on the peroxide value of plain skim milk, as well as one which prepared by using PUP method both containing added oils. As it has been shown, there is a significant difference between PUP and control samples in terms of their peroxide values.

In the very first hours of UV exposure, the peroxide in the control rose from 0.925 to 119.88 (meq/kg oil), but had more modest linear-like increase up to 9 hours and then followed a falling pattern, while in the case of PUP, the peroxide value only increased up to 1.37 (meq/kg oil) after 18 hours. As can be seen, natural casein micelle nanocapsules showed extremely superior oxidative protection on highly unsaturated oils. This could be partially due to the suspension of oil droplets by proteins, as well as protection via encapsulation by casein nano-micelle, that itself reduces the oil reaction capability and its accessibility to oxidative factors. In addition, in protein emulsions, the excess of proteins in the bulk may scavenge metals and keep them away from the oil's access; this could happen for casein protein more strongly since it has a high capacity for bonding to divalent cations (such as Iron).

The antioxidant effect and free-radical scavenging capacity of casein have also recently been reported. The reason for these characteristics is to some extent attributed to the presence of free thiol groups in kappa-casein. Previously, some reported that the re-assembled casein micelles (rCM), due to their having aromatic side groups and double bonds, have a good protective effect on vitamin D2 against ultraviolet radiation. In fact, it is likely that casein, because of absorbing or scattering much of the light, prevents the UV light from reaching the hydrophobic core (like rapeseed oil or any ω-3 oils containing edible oils).

In conclusion, the present invention revealed that skim milk has a high level of UV light absorption over the range 220-380 nm. As is understood in the art, proteins, particularly casein micelles, are the major structural ingredient in milk; thus, one can consider that casein micelles are able to absorb ultraviolet lights and prevent these destructive rays from reaching the hydrophobic core. As mentioned hereinabove, skim milk containing rapeseed oil (0.54%) was chosen as the control sample so that this experiment could be an appropriate criterion for investigating the oxidation differences in encapsulated and untreated samples.

The presence of a considerable difference in the oxidative stability was possibly due to the oxygen accessibility in encapsulated and untreated oil droplets. Moreover, casein micelles can form a thicker interfacial layer (10 nm) in oil-in-water emulsion droplets than whey proteins (-2 nm). It has also been reported that casein has higher oxidative stability in pH 3 compared to serum and soy proteins. But, the emulsions, which were stabilized by casein in pH 7 (with and without the use of transglutaminase enzyme), did not show a good oxidative stability in 8 days of incubation at 55° C. Therefore, alkaline pH along with ultrasound was more accessible to the hydrophobic areas of casein micelle, where oil particles could penetrate the interior parts of the casein micelles. As a result, it created reasonably high protection for unsaturated oils against UV light (at neutral pH about 6.7).

While the present invention has been illustrated by the description of the embodiments thereof, and while the embodiments have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the breadth or scope of the applicant's concept. Furthermore, although the present invention has been described in connection with a number of exemplary embodiments and implementations, the present invention is not so limited but rather covers various modifications and equivalent arrangements, which fall within the purview of the appended claims.

What is claimed is:

1. A method for nanoencapsulation of hydrophilic compounds comprising:
preparing a first dispersion containing therein a plurality of hydrophilic compositions;
preparing a second dispersion containing therein a plurality of casein micelles;
adjusting the pH of said second dispersion from a first pH to a second pH;
admixing said first dispersion and the adjusted second dispersion, forming an admixture thereof; and
sonicating said admixture,
whereby the sonicated admixture contains a plurality of casein micelles with said hydrophilic compositions encapsulated therein.

2. The method according to claim 1, wherein said plurality of hydrophilic compositions comprise at least two types of said hydrophilic compositions.

3. The method according to claim 1, wherein said plurality of hydrophilic compositions are selected from the group consisting of a drug, a nutraceutical, a vitamin, a cosmetic compound, and combinations thereof.

4. The method according to claim 1, wherein said plurality of hydrophilic compositions comprise a nutraceutical, where said nutraceutical is selected from the group consisting of ω-3 fatty acids.

5. The method according to claim 1, wherein said step of adjusting said second dispersion comprises increasing the pH toward alkalinity.

6. The method according to claim 5, wherein said second pH is in the range of about 7.5 to about 11.

7. The method according to claim 6, wherein said second pH is in the range of about 7.5 to about 8.

8. The method according to claim 1, further comprising:
after said sonicating, modifying the pH of the sonicated admixture.

9. The method according to claim 8, wherein said step of modifying said sonicated admixture comprises decreasing the pH toward neutrality to a third pH.

10. The method according to claim 9, wherein said third pH is in the range of about 6.5 to about 7.

11. The method according to claim 9, wherein said third pH is substantially equal to said first pH.

12. The method according to claim 1, wherein said plurality of casein micelles contain natural casein micelles.

13. The method according to claim 12, wherein said plurality of casein micelles contain about 1% to about 5% natural casein micelles.

14. The method according to claim 1, wherein said sonication of said admixture is for about 2 to about 4 minutes.

15. A nanoencapsulation comprising:
a casein micelle encapsulating a hydrophobic compound therein produced pursuant to the steps of claim 1.

16. The nanoencapsulation according to claim 15, wherein said casein micelle is a natural casein micelle.

17. The nanoencapsulation according to claim 15, wherein said hydrophilic composition is selected from the group consisting of a drug, a nutraceutical, a vitamin, a cosmetic compound, and combinations thereof.

18. The nanoencapsulation according to claim 15, wherein said hydrophilic composition is a nutraceutical, where said nutraceutical is selected from the group consisting of ω-3 fatty acids.

19. The nanoencapsulation according to claim 15, wherein said nanoencapsulation of said hydrophilic composition inside a casein micelle has an average diameter of about 100 nm to about 350 nm.

20. The nanoencapsulation according to claim 19, wherein said nanoencapsulation has an average diameter of about 200 nm to about 350 nm.

21. The nanoencapsulation according to claim 20, wherein said nanoencapsulation has an average diameter of about 250 nm to about 350 nm.
